(19) United States
(12) Patent Application Publication
(10) Pub. No.: US 2023/0241057 A1
(43) Pub. Date: Aug. 3, 2023

(54) COMPOUNDS FOR THE PREVENTION, TREATMENT AND DIAGNOSIS OF THROMBI

(71) Applicants: INSERM (INSTITUT NATIONAL DE LA SANTÉ ET DE LA RECHERCHE MÉDICALE), Paris (FR); UNIVERSITÉ DE PARIS, Paris (FR); UNIVERSITÉ PARIS XIII PARIS-NORD, Villetaneuse (FR); CARDIOVASCULAR LAB S.P.A, MILAN (IT)

(72) Inventors: Giuseppina CALIGIURI, Paris (FR); Antonino NICOLETTI, Paris (FR); Marta ANTONUCCI, Milano (IT); Charles SKARBEK, Paris (FR)

(21) Appl. No.: 17/999,910

(22) PCT Filed: May 27, 2021
(86) PCT No.: PCT/EP2021/064257

§ 371 Date: Nov. 25, 2022

(30) Foreign Application Priority Data
May 28, 2020 (WO) PCT/IB2020/000507

(51) Int. Cl. A61K 31/496; A61K 31/5415; A61K 31/4025; C07K 16/18; A61K 31/165; A61K 31/343; A61K 31/787; A61P 7/02; A61L 31/10; A61L 31/16

(52) U.S. Cl. CPC A61K 31/496; A61K 31/5415; A61K 31/4025; C07K 16/18; A61K 31/165; A61K 31/343; A61K 31/787; A61P 7/02; A61L 31/10; A61L 31/16; A61L 2420/06; A61L 2300/42

(57) ABSTRACT

The present invention relates to compounds for medical use in the treatment or in the prevention or in the diagnosis of arterial or venous thromboembolism.

### FIELD OF THE INVENTION

The present invention finds application in the medical field and in particular in the prevention, treatment and diagnosis of thrombus.

### BACKGROUND OF THE INVENTION

Endovascular thrombi (clots) require a rapid and effective removal, in order to restore blood flow to the affected organ in the shortest possible time.

Thrombolytic drugs generally act slowly and often fail, due to the local generation of neutrophil extracellular traps (NETs) because the scaffold made of NETs extracellular chromatin modifies and supplements the fibrin matrix.

Mechanical thrombectomy devices can remove the clot in a few minutes and have been shown to be cost-effective. However, the metal struts of thrombectomy devices can fail to grasp clots adhering to the arterial wall, leading to the need for repeat passages, with the risk of damaging the artery.

Furthermore, the device cannot retain small clot fragments, which can detach from the clot during mechanical removal attempts, leading to secondary embolization and dramatic complications of the procedure.

The article of Zandleh M. et al. (“Interfacing DNA and Polydopamine Nanoparticles and its Applications”, 9 Sep. 2020, https://doi.org/10.1002/ppsc.202000208) discloses the preparation of polydopamine-DNA conjugates via covalent attachment with amino- or thio-modified DNA under specific conditions of low pH and the presence of polyvalent metal ions.

### SUMMARY OF THE INVENTION

The inventors of the present patent application have surprisingly found that molecules bind to the neutrophil extra-cellular traps (NETs) within a thrombus. In particular, the invention is defined by claims.

### DETAILED DESCRIPTION OF THE INVENTION

According to a first object, there are disclosed ligands of the Neutrophil extra-cellular traps (NET) for use as a medicament in the prevention, in the treatment or in the diagnosis of arterial or venous thromboembolism.

According to a second object, there is disclosed a substrate comprising the ligands of the invention.

As per an embodiment, said substrate is in the form of a coating.

According to a third object of the invention, it is disclosed a process for the preparation of a coating comprising the disclosed ligands.

According to a fourth object, there are disclosed devices comprising a portion coated with the molecules or the coating of the invention.

According to a fifth object, there is disclosed a method for the prevention or for the treatment or for the diagnosis of arterial or venous thromboembolism comprising the use of the ligands of the invention.

According to another object, there is disclosed the use of the ligands of the invention for the adhesion to the thrombus, more in particular to the chromatin composing the dense mesh of neutrophil extra-cellular traps.

As per an embodiment, there is disclosed the use of the ligands of the invention for the adhesion and the removal of thrombus.

According to a further object, there is disclosed the use of the ligands of the invention in the field of interventional neuroradiology.

As per a still further object, there is disclosed the use of the ligands of the invention in the field of percutaneous cardiovascular intervention.

For the purposes of the present invention “ligand” shall be intended as a molecule capable of binding to a target.

For the purposes of the present invention, the “target” of a “ligand” is represented by neutrophil extra-cellular traps (NETs).

The presence of neutrophil extra-cellular traps (NETs) in arterial and venous thromboembolic matter has been already disclosed (J Thromb Haemost. 2014 June; 12(6):860-70; Ann Neurol. 2017 August; 82(2):223-232; Semin Thromb Hemost. 2019 February; 45(1):86-93; and several other published articles).

NETs are represented by extra-cellular meshes composed of decondensed chromatin, comprising DNA and the associated proteins, bearing citrullinated histones and the tight binding of neutrophil granular proteins, mainly represented by myeloperoxidase, neutrophil elastase and cathepsin G.

In a particular embodiment of the invention, within the NETs the ligands do target chromatin (DNA and associated proteins, such as topoisomerases).

In another embodiment of the invention, within the NETs the ligands do target citrullinated histones.

In a further embodiment of the invention, within the NETs the ligands do target the neutrophil granule proteins.

More in general, for the purposes of the present patent application, the targets of the invention ligands may be represented by other compounds that can be found within a thrombus, while under normal conditions they are found within the leucocytes.

In the following description they are referred to as leucocyte-derived nuclei-cytoplasmic compounds.

Said compounds may generally be represented by nuclear compounds, such as the above-mentioned chromatin, or by cytoplasmic compounds, such as the granules and protease granules.

For the purposes of the invention, the above disclosed targets are not in the flowing blood and are within a vascular thrombus.

According to a first object of the invention, the disclosed ligands are described for the medical use in the prevention, in the treatment and in the diagnosis of arterial or venous thromboembolism.

Within the present invention, said ligands do not include digoxigenin and distamycin for interventional neuroradiology.

For the purposes of the present invention, said ligands do not include digoxigenin and distamycin for interventional neuroradiology, wherein the use in the interventional neuroradiology includes thrombectomy intervention in the anterior, middle and posterior cerebral artery.

As used herein term “diagnosing” refers to classifying a disease or a symptom, determining a severity of the disease, monitoring disease progression, forecasting an outcome of a disease and/or prospects of recovery.

As used herein, the terms “treating” or “treatment” refer to both prophylactic or preventive treatment as well as curative or disease modifying treatment, including treatment of subject at risk of contracting the arterial or venous thromboembolism or suspected to have contracted arterial or venous thromboembolism as well as subject who are ill or have been diagnosed as suffering from an arterial or venous thromboembolism or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of an arterial or venous thromboembolism or recurring arterial or venous thromboembolism, or in order to prolong the survival of a subject beyond that expected in the absence of such treatment.

As used herein, the term “thromboembolism” refers to the formation in a blood vessel of a clot (thrombus) that breaks loose and is carried by the blood stream to plug another vessel. In the context of the invention, the clot may plug a vessel in the lungs (pulmonary embolism), brain (stroke), gastrointestinal tract, kidneys, or leg.

As used herein, the term “venous thromboembolism” refers to a blood clot (thrombus) that forms within a vein. A common type of venous thrombosis is a deep vein thrombosis (DVT), which is a blood clot in the deep veins of the leg. If the thrombus breaks off (embolizes) and flows towards the lungs, it can become a pulmonary embolism (PE), a blood clot in the lungs. Various other forms of venous thrombosis also exist; some of these can also lead to pulmonary embolism.

As used herein, the term “arterial thromboembolism” refers to the formation of a thrombus within an artery. In most cases, arterial thrombosis follows rupture of atheroma (a fat-rich deposit in the blood vessel wall), and is therefore referred to as atherothrombosis. Arterial embolism occurs when clots then migrate downstream, and can affect any organ.

For the purposes of the present invention conditions referring to secondary embolisms are included as well.

In particular, said conditions shall be intended as comprising both a condition secondary to embolization/embolism and a distal embolization occurring during the procedure of thrombectomy.

Within the present invention, the prevention of arterial or venous thromboembolism comprises the prevention of a condition selected from the group comprising distal embolization and pulmonary embolism.

In a non-limiting way, said distal embolization conditions may occur for instance in rheolytic/rotational thrombectomy procedures as well as during percutaneous procedures such as cardiac valve implantation, carotid stenting and peripheral artery angioplasty/stenting.

In a further embodiment, the ligands of the neutrophil extra-cellular traps (NETs) (or leucocyte-derived nuclei-cytoplasmic compounds) for medical use according to the invention, wherein said treatment comprises the treatment of a condition selected in the group comprising: occlusive vascular conditions.

As used herein, the term “occlusive vascular condition” refers to vascular disorder-involving blockage in an organ. In a particular embodiment, the occlusive vascular condition can be the blockage in the carotid or femoral arteries, including the iliac artery, also known as peripheral arterial occlusive condition.

In another embodiment, the ligands of the neutrophil extra-cellular traps (NETs) (or leucocyte-derived nuclei-cytoplasmic compounds) for medical use according the invention, wherein said occlusive vascular condition leads to an acute organ ischemia.

Ischemia refers to a reduction or interruption of the blood flow to one or more organs.

As used herein, the term “acute organ ischemia” refers to a reduced organ function that occurs rapidly in days or weeks.

In a non-limiting way, said pulmonary embolism conditions may occur for instance in deep vein thrombosis.

For the purposes of the present invention, the disclosed prevention and treatment of an arterial or venous thromboembolism shall also be intended to cover all those conditions wherein the disclosed ligands improve the performance of a device in the removal of a thrombus or a thrombus fragment, with particular reference to preventing thrombus fragments from detaching.

Within the present invention, the treatment of arterial or venous thromboembolism comprises the treatment of a condition selected from the group comprising organ infarction, thrombosed dialysis grafts, occluded vein grafts, in-stent thrombosis, venous thromboembolism.

Within the present invention, diagnosis of arterial or venous thromboembolism comprises the use of contrast agents suitably linked to the ligands of the invention.

Suitable contrast agent may comprise for instance iodinated compounds or Gadolinium compounds.

For the purposes of the present invention, ligands may include:
DNA ligands, and
Topoisomerases, and
Histone ligands.

In particular, DNA ligands may include:
Intercalating ligands,
Groove-binding ligands, and
DNA alkylating agents.

Intercalating ligands may be selected in the group comprising: ciprofloxacine, methylene blue, berberine, proflavin, daunomycin, doxorubicin and thalidomide.

Groove-binding ligands may be minor groove-binding ligands or major groove-binding ligands, selected in the group comprising: sulindac, netropsin, tallimustine, hairpin polyamides, bis(benzimidazoles), aureolic acids and bisquaternary ammonium heterocycles.

DNA alkylating agents may be selected in the group comprising: all bis-chloroethylamine or bis-bromoethylamine derivatives such as chlorambucil, norchlorambucil, bendamustin, bromo benzoic mustard or melphalan.

Busulfan, N-(2-(2-(2-azidoethoxy)ethoxy)ethyl)-5-(bis(2-chloroethyl)amino)benzofuran-2-carboxamide (MBF) and 4-[Bis-(2-bromoethyl)amino]benzoic acid may also be selected as the DNA alkylating agent.

For the purposes of the present invention, there are disclosed ligands having a DNA alkylating activity having one of the following general structure:

I) ω-azido polyethylene glycol methanesolfonate derivatives

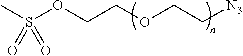

wherein n is from 1 to 6.

II) a 4-(bis(2-haloethyl)amino)phenyl moiety joined to a ω-azido polyethylene glycol chain with a carboxamido spacer

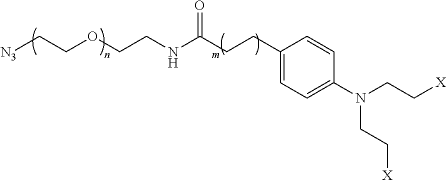

wherein

n is from 1 to 6

m is from 0 to 6

X is halogen selected from Cl, Br, I.

III) a benzoheterocycle such as benzo[b]furan, indole, N-alkylindole, indazole, benzo[b]thiophene bearing at their 5-position a bis(2-haloethyl)amino) moiety and joined by carboxamide group to a ω-azido polyethylene glycol spacer

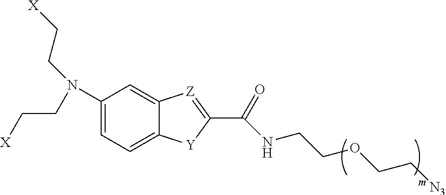

wherein

m is from 1 to 6.

X is halogen selected from: Cl, Br, I)

Z is —CH— or —N—,

Y is —O—, —S—, —NH or N—C₁₋₆alkyl alkyl chain.

Histone ligands may be anti-histone antibodies or fragments thereof, such as for instance: Fab, F(ab′)2, Fab2, scFv.

In a preferred embodiment, the ligands are selected from the group comprising: Digoxigenin, Distamycin, Ciprofloxacin, Thioridazine, Netropsin, Trabectedin, Sulindac, Piperaquine, Atabrine (Mepacrine), Mitonafide, Chloroquine, Amsacrine, Indomethacin, Methylene blue, berberine, proflavin, daunomycin, doxorubicin, thalidomide, tallimustin, hairpin polyamides, bis(benzimidazole), aureolic acids and bisquaternary ammonium heterocycles) and derivatives of any one of the above.

In a still preferred embodiment, the ligands are represented by digoxigenin and distamycin.

In a further embodiment, digoxigenin and distamycin are used for the treatment of a condition selected from the group comprising but not limited to: organ infarction, thrombosed dialysis grafts, occluded vein grafts, in-stent thrombosis, arterial and venous thromboembolism, except thrombectomy procedures in the domain of interventional neuroradiology.

For the purposes of the present invention, derivatives of the above-mentioned compounds shall be intended as ligands as well.

Derivatives shall include as isomers, enantiomers, azido-derivatized compounds.

Derivatives shall also include esters.

Flexible and rigid spacers can be comprised between the ligand and the derivatized group as to increase the accessibility of the ligand to the target within the thrombi.

According to an embodiment of the present invention, the above disclosed ligands are within a substrate.

For the purposes of the present invention, the ligands may be linked to said substrate.

In particular, the linkage may occur via a linker, as disclosed more in detail here below.

In an embodiment of the present invention, the substrate may be in the form of a coating, that is to say that it can form a coating onto a surface.

In another embodiment of the present invention, there may be provided a secondary coating.

Said secondary coating may be an over-coating and may have a protective effect with respect to the coating underneath.

For the purposes of the present invention, the secondary coating or the over-coating may comprise a saccharide or a polysaccharide or a mixture of saccharides or of polysaccharides.

In a preferred embodiment, the over-coating is a mannitol over-coating.

Other secondary coatings may be used if suitable within the purposes of the present invention.

Said substrate and said coating comprising a ligand of the invention do represent a second object of the present invention.

According to a third object of the invention, it is disclosed a process for the preparation of a coating comprising a ligand of the invention.

In particular, said process comprises a first step for the preparation of a solution or of a suspension of the substrate.

A substrate may be represented by a suitable polymer or co-polymer.

Non-limitative examples of substrates are represented by polydopamine, PEG-bis-amine and copolymer polydopamine and PEG-bis-amine.

A further example of suitable substrate comprises for instance a sequential layering of polymers and/copolymers, independently one another; a non-limitative example of substrate is represented by a sequential layering of polydopamine and PEG-bis-amine.

In a subsequent step, a coating is formed on a device or on a portion of a device by dip-coating of the device or of the device portion into the substrate solution or suspension.

For linking to the coating, a ligand of the invention shall be conveniently derivatized.

Derivatization technique is suitably selected in view of the ligand and may comprises:
NHS (N-hydroxysuccinimidyl ester) derivatization. NHS derivatization is particularly suitable for PEG-bis-amine substrates;
Azide derivatization. Azide derivatization include the insertion of an azide group via a suitable linker.

Other methods include:
methoxy PEG amine (NH₂-PEG-COOH) for subsequent EDC/SNHS (N-ethyl-N′-(3-(dimethylamino)propyl)carbodiimide/N-hydroxysuccinimide reaction for linking antibody and antibody fragments, like Fc fragments; and
click-chemistry via suitable bifunctional reagent, like DBCO-PEG4-amine.

In a preferred embodiment of the invention, the azide derivatization includes the insertion of an azide group via a suitable linker.

For the purposes of the present invention, the linker may be:
a polyethylene linker,
a polyether linker.

In particular, as shown in FIG. 18A, a polyethylene linker may have the following structure:

—(O-CH₂−CH₂)ₙ—

wherein n is from 3 to 5.

In particular, as shown in FIG. 18B, a polyether linker may have the following structure:

-(—CH₂)ₘ—O—(CH₂)ₙ—

wherein m and n are independently 3 to 7.

In a particular embodiment, as shown in FIG. 19, the linker may have the following general formula (I):

-[(—CH₂)ₙ—O—]_{z}-(CH₂)ₘ—N₃,

wherein

n is from 1 to 10, preferably from 2 to 6 and more preferably from 2 to 4,

z is from 1 to 6, preferably from 1 to 4 and more preferably from 1 to 3,

m is from 1 to 10 and preferably from 2 to 5.

Preferred linkers according to the present invention are:

| Reference | Structure |
|---|---|
| Pipe-1 | 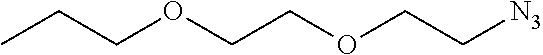 |
| Pipe-2 | 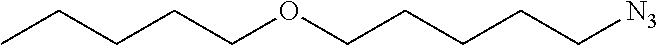 |
| Pipe-3 | 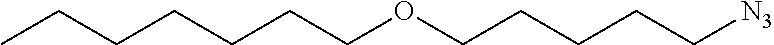 [SMILES: CCCCCCCOCCCCCN=[N+]=[N-]] |
| Pipe-4 | 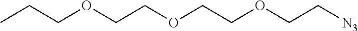 |
| Pipe-5 | 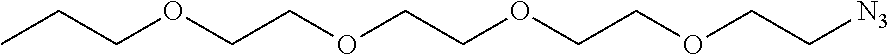 [SMILES: CCCOCCOCCOCCOCCN=[N+]=[N-]] |

According to a preferred embodiment of the invention, the azide derivatives of the ligands of the invention comprise: N-(2-(2-(2-azidoethoxy)ethoxy)ethyl)-5-(bis(2-chloroethyl)amino)benzofuran-2-carboxamide (benzofuran azide mustard, MBF), 4-[Bis-(2-bromoethyl)amino]benzoic acid (BBM), N-(2-(2-(2-azidoethoxy)ethoxy)ethyl)-3-(4-(bis(2-chloroethyl)amino)phenyl)propanamide (PPM, norchlorambucil-azide), N-(2-(2-(2-azidoethoxy)ethoxy)ethyl)-4-(4-(bis(2-chloroethyl)amino)phenyl)butanamide (chlorambucil-azide), 4-(4-(4-((5-azidopentyl)oxy)butyl)piperazin-1-yl) chloroquinoline (Piperaquine-azide), 4-[Bis-(2-bromoethyl)amino]benzoic acid (azobenzene-azide, BBM), busulfan-azide (MsA).

According to a particular embodiment of the invention, polydopamine can act also as a ligand; therefore, the substrate comprising polydopamine may need or may need not to include additional ligand(s).

In an embodiment of the present invention, there may be a secondary coating.

Said secondary coating may be an over-coating.

Said secondary coating may be particularly useful when the substrate comprises a ligand with less selectivity towards chromatin.

Said secondary coating may be particularly useful when polydopamine is used as the substrate forming the coating or as a ligand.

For the purposes of the present invention, the over-coating is formed on a device or on a portion of a device by dip-coating of the device or of the device portion into a solution or suspension of the secondary coating.

Alternatively, the over-coating may be applied to the device or to a portion of the device by applying a solution or a suspension of the secondary coating.

A solution or a suspension of the secondary coating may be prepared according to methods known in the art.

According to a fourth object, there are disclosed devices coated with the ligands of the invention.

In a preferred embodiment, said ligands are within a substrate or coating as per the above disclosure.

For the purposes of the present invention, a device may be completely or partially coated with the disclosed coating.

For the purposes of the present invention, a device may be completely or partially additionally coated with the secondary coating of the invention.

Said secondary coating may be a water-soluble or biodegradable over-coating.

As per an embodiment, a device may include:
thrombectomy device,
flowretriever,
filters,
embolic protection devices,
distal protection devices, including balloon angioplasty,
inferior vena cava filters.

As per another embodiment, a device may include devices for: rheolytic/rotational thrombectomy procedures, percutaneous procedures such as: coronary thrombectomy, cardiac valve implantation, carotid stenting, peripheral artery angioplasty/stenting, inferior vena cava filters, venous thromboembolism, percutaneous cardiovascular procedures;

According to a fifth object of the invention, there is disclosed a method for the prevention and the treatment of venous or arterial thromboembolism in a subject.

In particular, said conditions shall be intended as comprising both a condition secondary to embolization/embolism and a distal embolization occurring during the procedure of thrombectomy.

Within the present invention, the prevention of arterial or venous thromboembolism comprises the prevention of a condition selected from the group comprising distal embolization and pulmonary embolism.

In a non-limiting way, said distal embolization conditions may occur for instance in rheolytic/rotational thrombectomy procedures as well as during percutaneous procedures such as cardiac valve implantation, carotid stenting and peripheral artery angioplasty/stenting.

In a non-limiting way, said pulmonary embolism conditions may occur for instance in deep vein thrombosis.

In particular, the method of the invention comprises the steps of contacting a thrombus or a fragment thereof with a substrate or a coating according to the invention.

In particular, said coating coats a portion of a device or a device according to the invention.

According to the embodiment disclosing the secondary coating, said secondary coating has dissolved so that the coating underneath can be contacted with the thrombus of a fragment thereof.

In a preferred embodiment of the invention, it is disclosed the treatment or the prevention of massive stroke, pulmonary embolism, myocardial infarction, intestinal infarction, acute limb ischemia.

As per an embodiment of the invention, there is disclosed a method for the diagnosis of venous or arterial thromboembolism in a subject.

In particular, said method comprises the administration of a suitable contrast suitably linked to the ligands of the invention.

A suitable contrast agent may comprise iodinated compounds or Gadolinium compounds.

As per another object, there is disclosed the use of the ligands of the invention for the adhesion to the thrombus, more in particular to the neutrophil extra-cellular traps.

As per an embodiment, there is disclosed the use of the ligands of the invention for the adhesion and the removal of thrombus, more in particular, through the binding of the ligands to the extracellular chromatin.

According to a further object, there is disclosed the use of the ligands of the invention in the field of interventional neuroradiology.

As above reported, it is not disclosed the use of digoxigenin and distamycin in the field of interventional neuroradiology.

In particular, the use in the interventional neuroradiology includes intervention in the anterior, middle and posterior cerebral artery.

As per an embodiment, there is disclosed the use of the ligands of the invention in a field other than interventional neuroradiology, such as in the field of percutaneous cardiovascular intervention.

In particular, the use in said non-interventional neuroradiology fields include for instance: coronary atherothrombosis, carotid atherothrombosis, pulmonary embolism, myocardial infarction, intestinal infarction, acute limb ischemia.

The invention will be further illustrated by the following figures and examples. However, these examples and figures should not be interpreted in any way as limiting the scope of the present invention.

### FIGURES

FIGS. 1 to 13 and 22 to 28 show the structure of some of the ligands of the invention.

FIG. 14 shows the results obtained with the procedure of Example 1 obtained with fluorescence microscopy.

FIG. 15 reports the graph with data on the Integrated Density (Average Signal Density multiplied by the area).

FIGS. 16 and 17 show the results of the comparative assay on the platelet and chromatin binding properties of some of the ligands of the invention as compared to neutrophil extra-cellular traps (NETs) as in Example 2.

FIGS. 18 and 19 report the structure of some of the linkers of the invention.

FIG. 20 shows the results of the comparative assay on the platelet and chromatin binding properties of some of the linkers used for piperaquine as the ligand.

FIG. 21 shows the steps of the experiment according to Example 3.

FIG. 29 reports the structure of polydopamine.

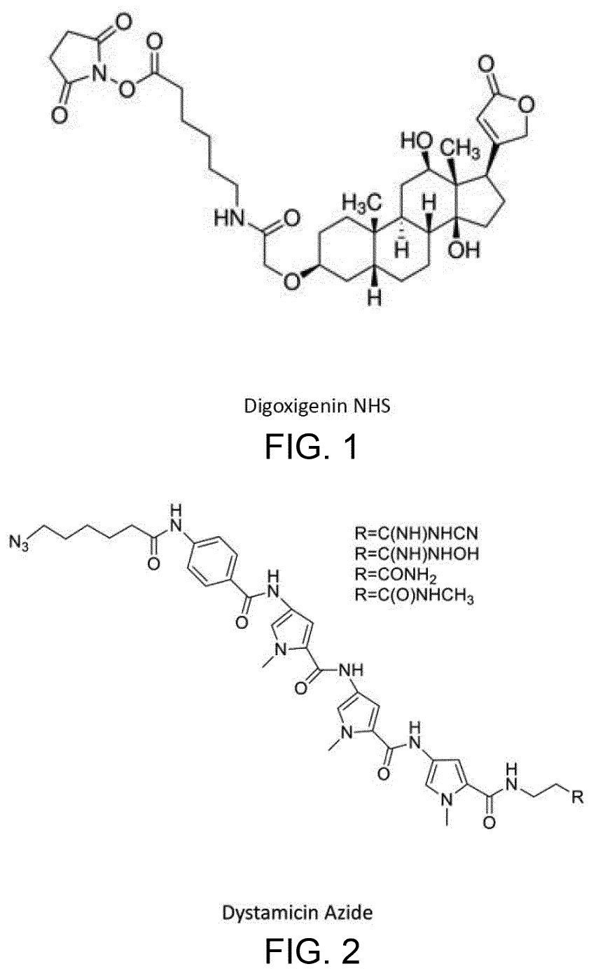

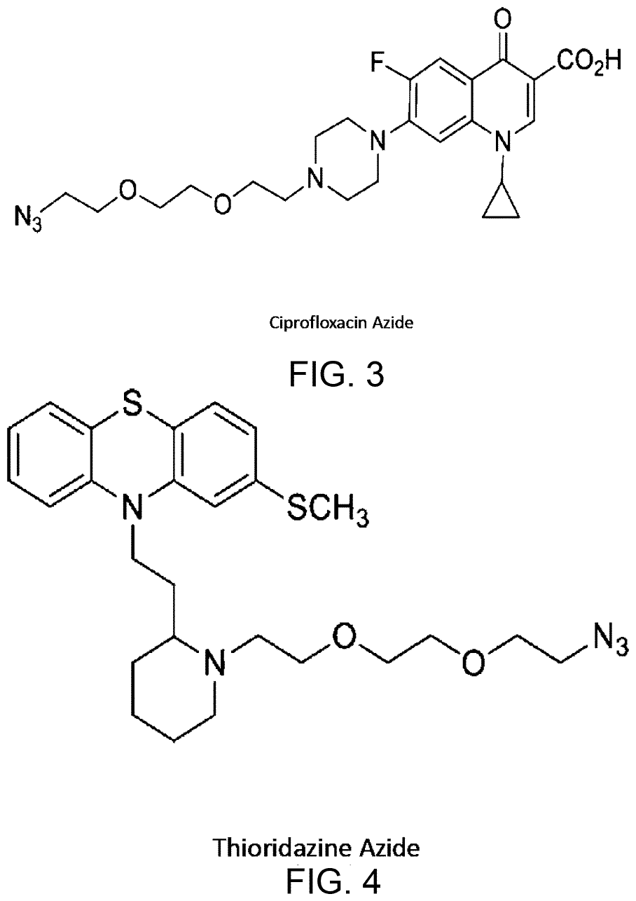

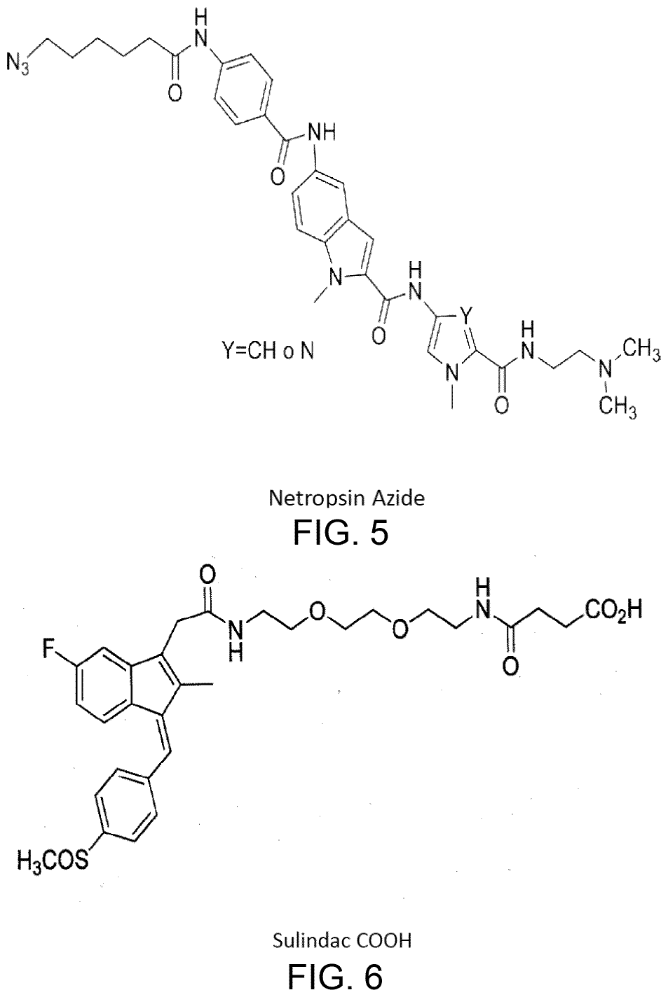

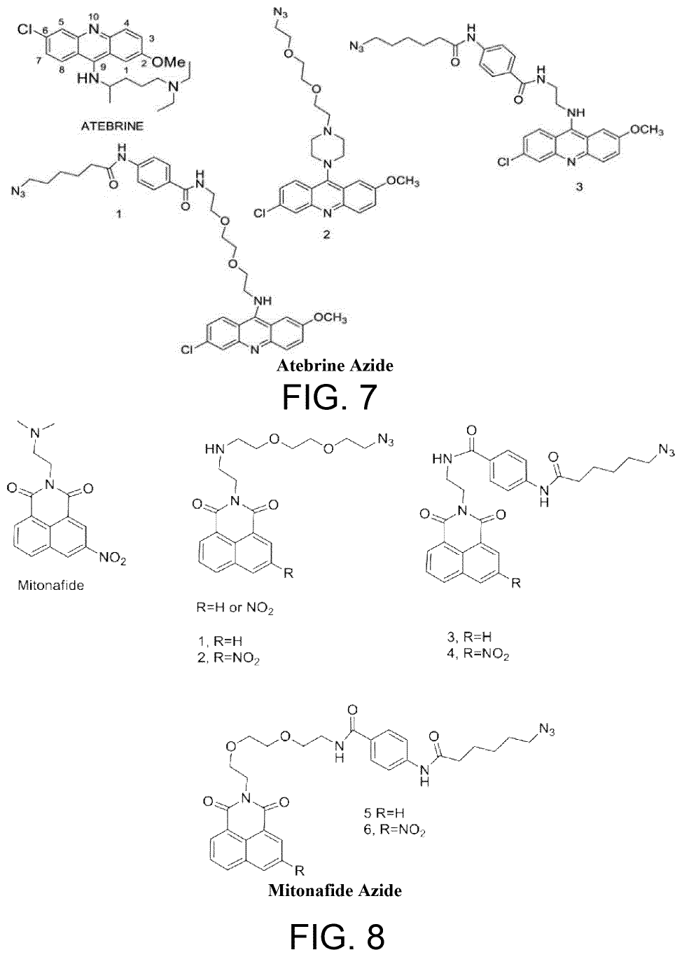

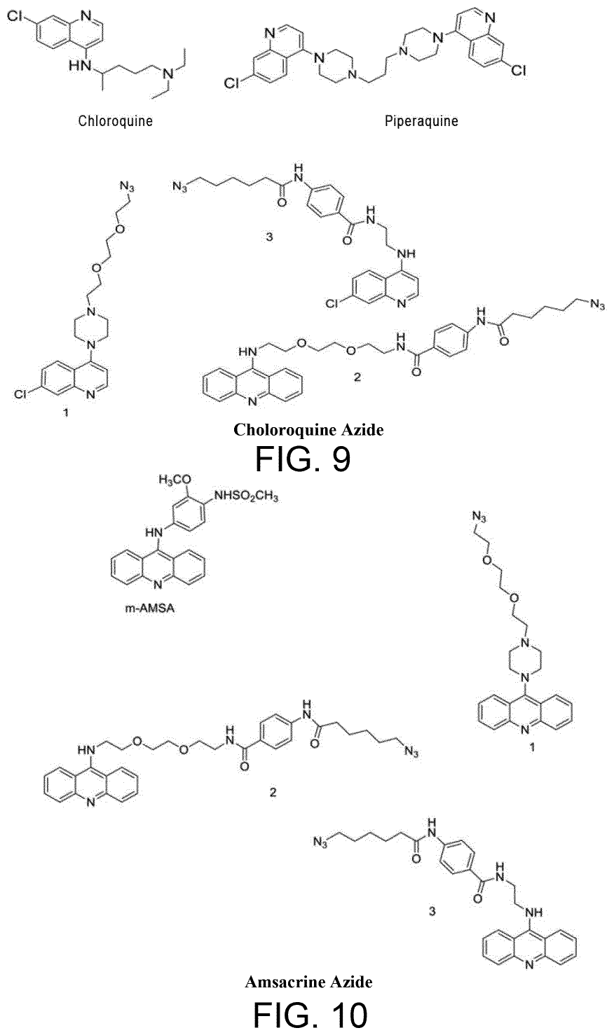

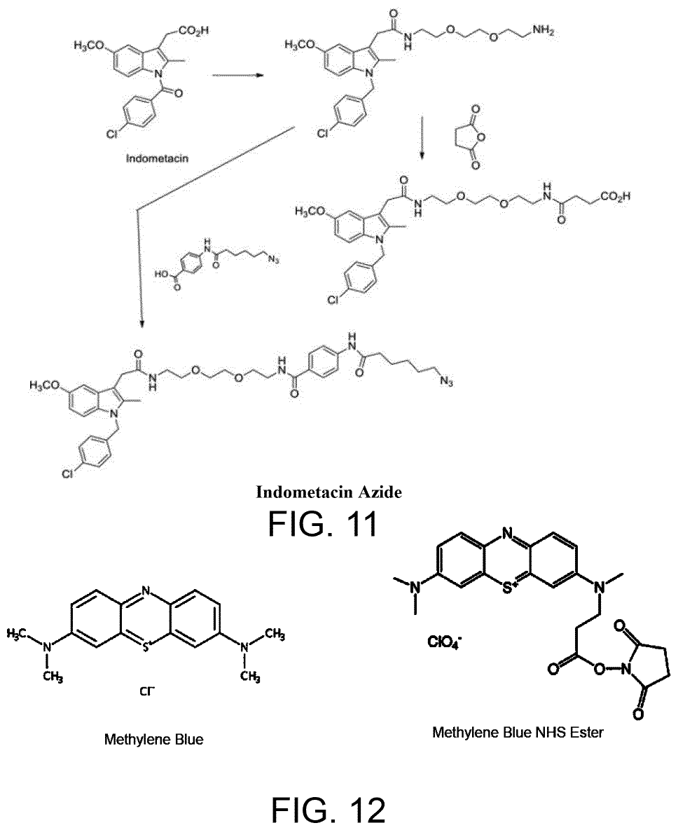

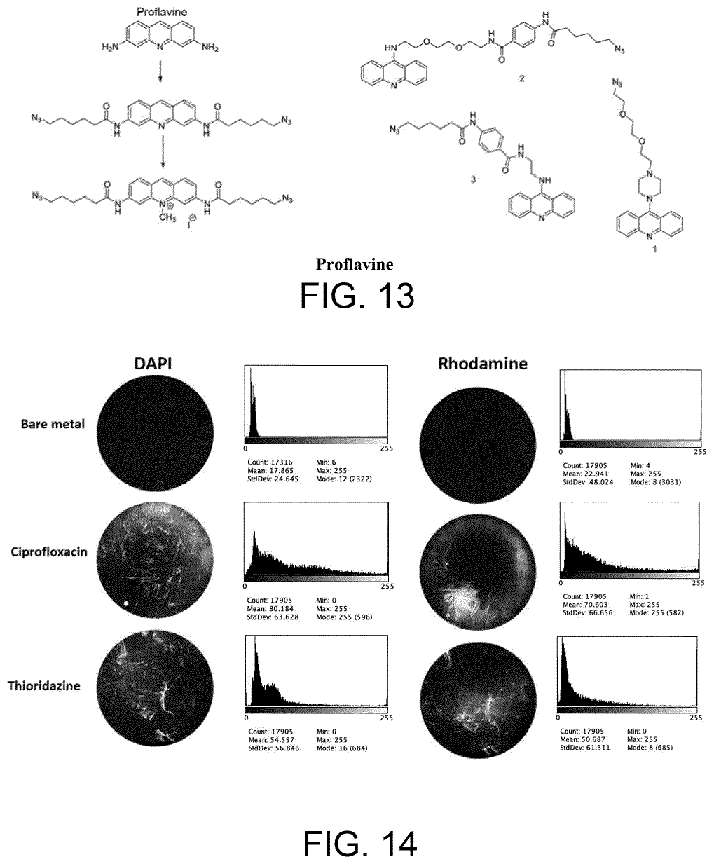

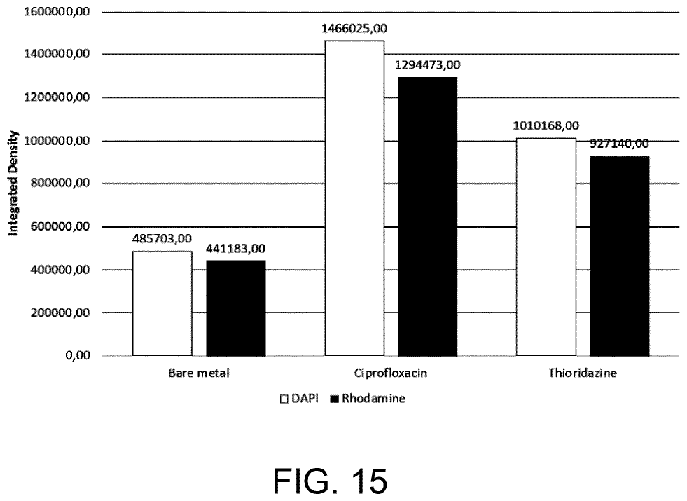

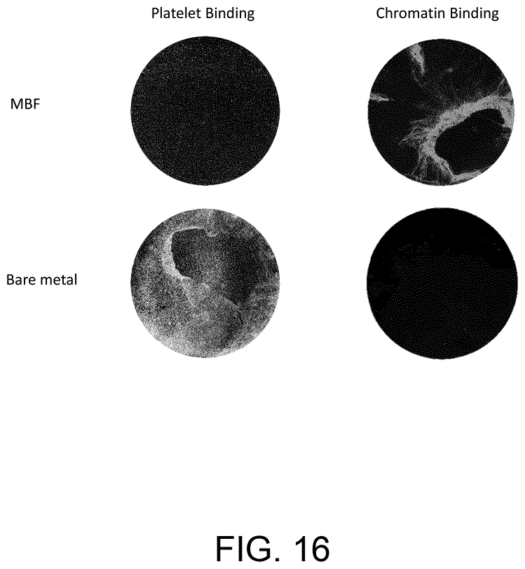

### EXAMPLE 1

### Analysis of Thrombus Material Captured by the Surfaces

Nitinol disks were dip-coated in three serial baths composed of

1. Polydopamine (1 mg/ml in tris buffer, pH 8.5, 22±2 hours)

2. DBCO-PEG 4-Amine

3. Azide-derivatized DNA-binding compounds (ciprofloxacin-N₃ or thioridazine-N₃)

A fresh thrombus was prepared with human whole blood additioned with PMA 100 nM and ionomycin 1 μM to promote strong leukocyte activation leading to the release of extracellular chromatin within the thrombus. Slice of the same thrombus were layered onto coated and bare metal disks in the wells of a 48-well plate, covered with human plasma and incubated at 37° C. on an orbital shaker for 5 minutes. The thrombus slices were mechanically removed using tweezers, the disk washed in PBS and stained with a solution made of Hoechst 33342 (1 μg/ml, to stain DNA) and Evans Blue (3%, to stain proteins) in PBS for 5 minutes. After careful washing, the presence of thrombus material-DNA (DAPI channel) and proteins (Rhodamine channel)—captured by the experimental surfaces was revealed by fluorescence microscopy. A representative image of the disk observed in the two channels is shown in FIG. 12. The image analysis was performed using the Image J open software. Integrated Density (Average Signal Density multiplied by the area) analysis showed that ciprofloxacin and thioridazine-coated surfaces consistently adhere to and capture more DNA and proteins from the thrombus.

### EXAMPLE 2

### Evaluation of the Chromatin and Platelet Binding

FIG. 16 reports the results of an experiment carried out in order to assay the selective binding properties exerted by different coating on blood platelets as compared to extracellular chromatin (NETs).

Experimental Setting

Nitinol disks are left untouched (bare metal) or coated with the tested chromatin ligands following a polydopamine, DBCO-PEG4-amine and azide derivative ligand sequential dip-coating layering procedure. Platelet adhesion (left panel) was evaluated by image analysis of the surface after staining with phalloidin coupled to AlexaFluor®488 (green fluorescence) after immersion of the disks for 10 min in blood withdrawn in PPAK (thrombin inhibitor providing a transient anticoagulation).

An example of disk displaying a low platelet binding (MBF coating, top) as compared to high binding to bare metal (bottom) is shown.

The extracellular chromatin binding was evaluated by applying for 3 minutes the disks on the bottom of wells in which fresh human neutrophils were stimulated with nigericin for 4 hours (PMID: 28574339; doi: 10.7554/eLife.24437), to promote the formation of neutrophil extracellular traps (NETs). Extracellular chromatin adhering to the disk surface was stained with the cell impermeant nuclear dye Sytox Green. An example of disk displaying a high (MBF coating, top) as compared to the low binding to bare metal (bottom) chromatin binding is shown.

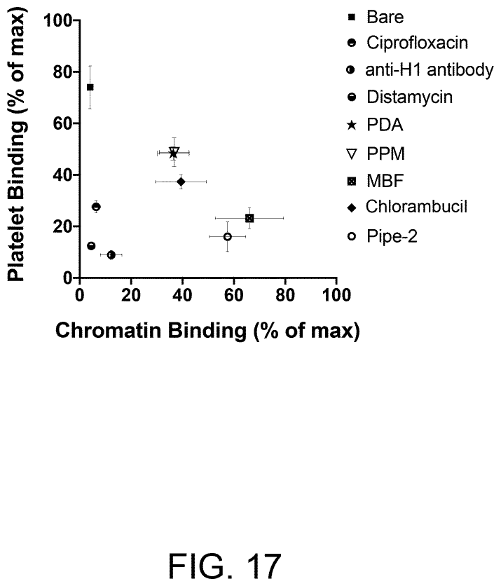

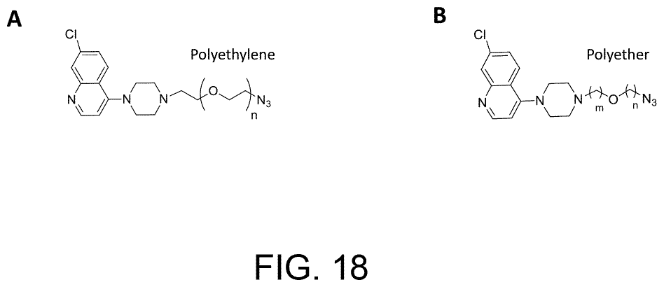

FIG. 17 reports the results of an experiment carried out in order to assay the binding properties exerted by different coating on blood platelets as compared to extracellular chromatin (NETs).

Example of the binding properties exerted by different coatings (N^{o}=6/coating) on blood platelets as compared to extracellular chromatin (NETs).

### Two-Way Dot Plot:

Each dot corresponds to the mean binding (% of max, mean±SEM) of chromatin (X-axis) and platelet (Y-axix) with the standard error bars parallel to the respective axis.

The best compounds are those in the right bottom quadrant, where the coatings display a high binding towards chromatin and poor binding towards blood platelets. At the opposite, the worst situation e.g. high platelet and low chromatin binding properties, is displayed in the left upper quadrant (bare metal surface).

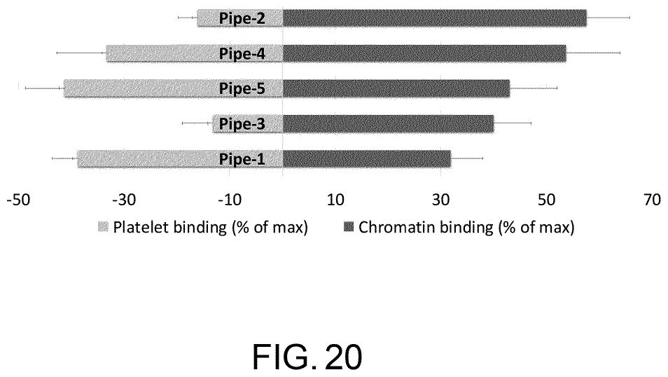

The results obtained are shown in FIGS. 17 and 20 and are reported in the following table:

| | Chomatin | Platelet | |
|---|---|---|---|
| | binding | binding | Chromatin/Platelet |
| Ligand | (% of max) | (% of max) | binding ratio |
| Bare metal | 4.03 ± 1.15 | 74.03 ± 11.02 | 0.05 |
| PDA | 36.39 ± 6.16 | 48.32 ± 2.53 | 0.75 |
| Ciprofloxacin | 23.63 ± 9.59 | 27.57 ± 3.22 | 0.86 |
| Distamycin | 12.26 ± 4.14 | 12.42 ± 1.72 | 0.99 |
| Anti-H1-antibody | 5.7 ± 1.71 | 8.89 ± 2.42 | 0.64 |
| PPM | 36.75 ± 5.78 | 48.8 ± 5.59 | 0.75 |
| MBF | 66.07 ± 13.21 | 23.18 ± 4.05 | 2.85 |
| Chlorambucil | 39.38 ± 9.9 | 37.32 ± 2.76 | 1.06 |
| Pipe-2 | 57.5 7.05 | 16 ± 5.78 | 3.59 |

### EXAMPLE 3

### Evaluation of the Effect of the Linker on the Binding Properties of a Given Ligand on Piperaquine

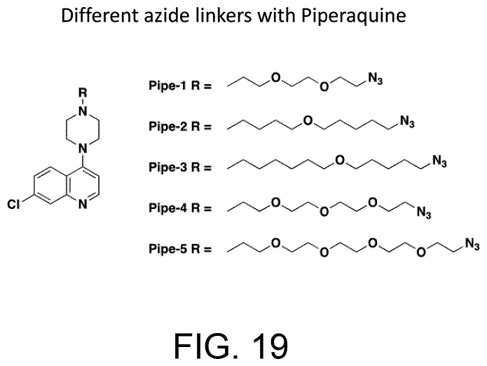

FIG. 19 shows the result of the experiment carried out to evaluate the effect of the linker on the binding properties of a given ligand on piperaquine.

Experimental Setting

Different linkers were combined with piperaquine and the resulting azide derivatives were used for coating nitinol disks (N=6 for each compound), following the described 3-step dip-coating procedure. Platelet adhesion (left panel) was evaluated by analyzing the surface and intensity of the phalloidin staining (green fluorescence) after immersion of the disks (N^{o}=6/coating) for 10 min in blood withdrawn in PPAK (thrombin inhibitor providing a transient anticoagulation). The binding of extracellular chromatin was evaluated by applying for 3 minutes the disks on the bottom of wells in which fresh human neutrophils were stimulated with nigericin for 4 hours, to promote the formation of neutrophil extracellular traps (NETs). Extracellular chromatin was stained with the cell impermeant nuclear dye Sytox Green.

The “mirror” bars in this figure indicate the platelet (left) as compared to the chromatin (right) binding (% of max, mean±SEM) binding for each of the displayed coatings, sorted (from the top to the bottom) according to the ability of each compound to clutch chromatin (best binding profile on the top).

### EXAMPLE 4

### Evaluation of the Clot-Device Detaching Time

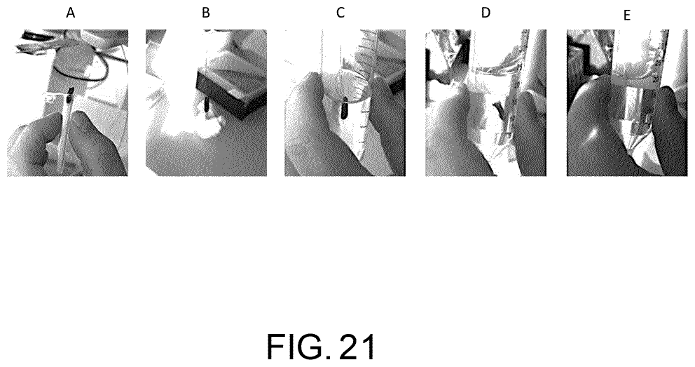

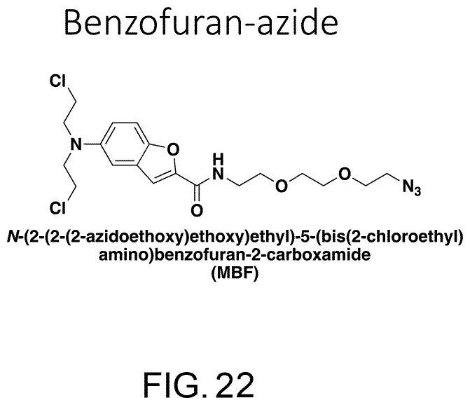

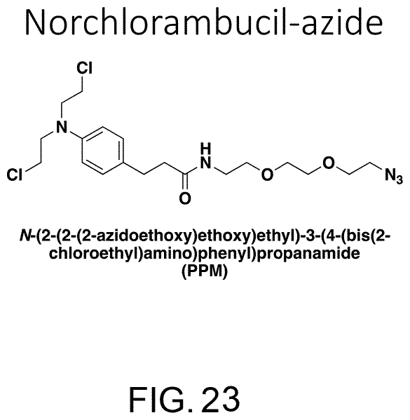

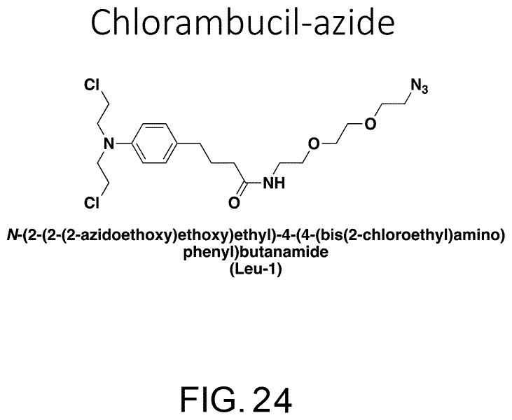

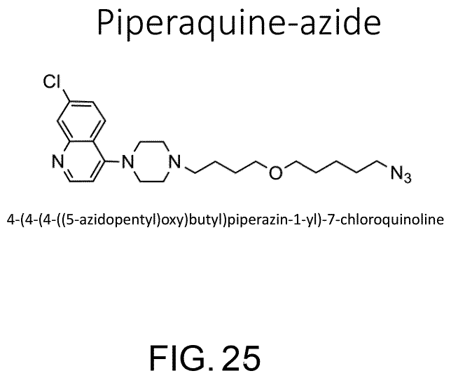

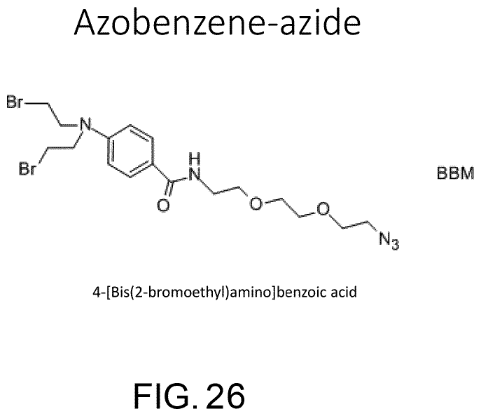

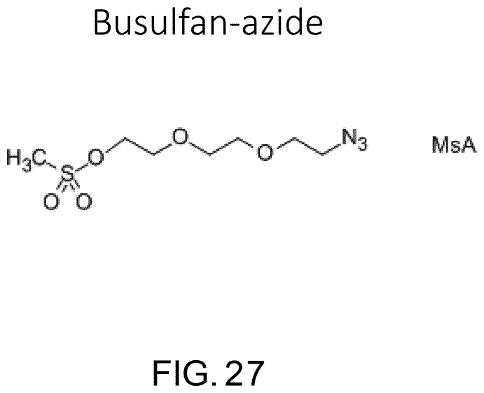

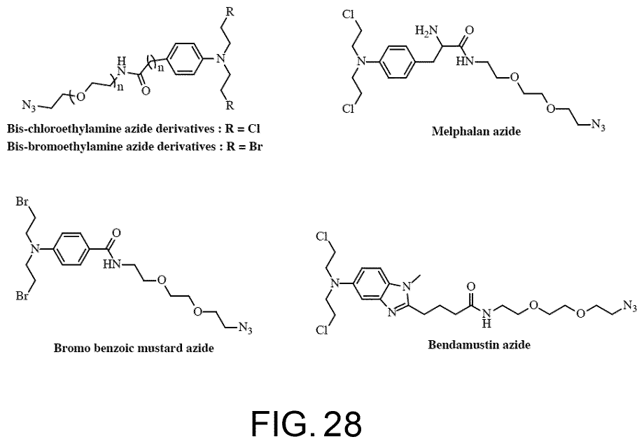

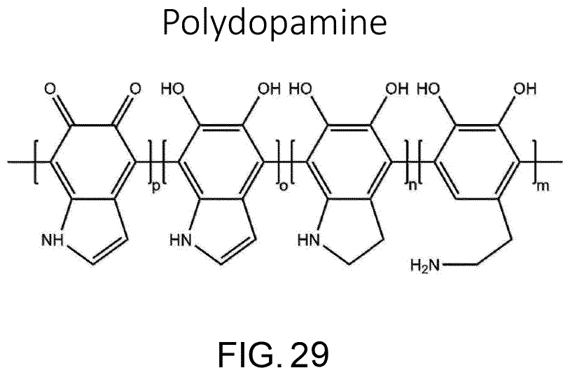

FIG. 21 shows the steps of the experiment carried out to evaluate the time required for the detaching of the captured clot from a stent-retriever.

For the purposes of this assay, several identical artificial clots are prepared using fresh human blood as previously described (PMID: 29695602; doi: 10.1136/neurintsurg-2017-013675) with the addition of nigericin to allow enrichment of NETs within the clot (PMID: 28574339; doi: 10.7554/eLife.24437). The clots are positioned at the bottom of individual small polypropylene tubes.

The experiment comprises the following phases:

1) Sheathed clinical grade thrombectomy devices (bare metal or coated with indicated ligands, following a sequential dip-coating layering procedure) are passed between the clot and the wall of the tube and deployed once they reach the bottom of the tube. Their ability to capture the clot is monitored by direct video recording (A).

2) The device is extracted from the tube and the occurrence of its tight adhesion to the clot is video-recorded (B).

3) The device with the adhering clot is transferred into a 50-ml tube containing saline (PBS) (C).

4) The device+adhering clot are vigorously agitated within the fluid (D).

5) The detachment of the clot is directly monitored by continuous video-recording (E).

The time required for the clot to detach from the device is quantified by blinded operators on the recorded videos (experiments in duplicate) and the data are as follows:

| Device type | Time delay to clot detachment (seconds) |
|---|---|
| Bare metal | 0 ± 0 |
| Piperaquine | 5 ± 2 |
| Chlorambucil | 15 ± 3 |
| Polydopamine | 23 ± 2 |

From the above description, the advantages of the present invention will be clear to the person skilled in the art.

State of the art strategies aim at dissolving the extracellular chromatin to allow effective pharmacologic thrombolysis.

Accordingly, the preset invention proposes, instead, to exploit the presence of the chromatin fibers to anchor the thrombus matter to ease its removal from the adhering vascular wall and stably retain its fragments to prevent secondary embolization.

1. Ligands of the neutrophil extra-cellular traps (NETs) for medical use in the treatment or in the prevention or in the diagnosis of arterial or venous thromboembolism, wherein said ligands do not include digoxigenin and distamycin for interventional neuroradiology.

2. Ligands according to the preceding claim, wherein said neutrophil extra-cellular traps (NETs) are within a thrombus.

3. Ligands of the extra-cellular chromatin for medical use according to the previous claim 1 or 2, wherein said ligands are selected from:
DNA ligands, and
Topoisomerases, and
Histone ligands.

4. Ligands of the extra-cellular chromatin for medical use according to the previous claim, wherein said DNA ligands comprise:
Intercalating ligands,
Groove-binding ligands, and
DNA alkylating agents.

5. Ligands of the extra-cellular chromatin for medical use according to the previous claim 3, wherein said intercalating ligands are selected in the group comprising: ciprofloxacine, methylene blue, berberine, proflavin, daunomycin, doxorubicin and thalidomide.

6. Ligands of the extra-cellular chromatin for medical use according to the previous claim 3, wherein said groove-binding ligands may be minor groove-binding ligands or major groove-binding ligands, selected in the group comprising: sulindac, netropsin, tallimustine, hairpin polyamides, bis(benzimidazoles), aureolic acids and bisquaternary ammonium heterocycles.

7. Ligands of the extra-cellular chromatin for medical use according to the previous claim 3, wherein said DNA alkylating agents have one of the following general structure:
I) ω-azido polyethylene glycol methanesolfonate derivatives 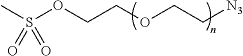
wherein n is from 1 to 6;
II) a 4-(bis(2-haloethyl)amino)phenyl moiety joined to a ω-azido polyethylene glycol chain with a carboxamido spacer 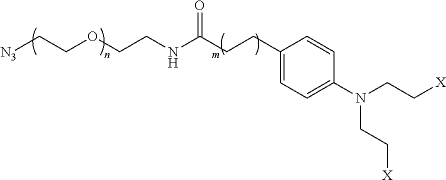
wherein
n is from 1 to 6
m is from 0 to 6
X is halogen selected from Cl, Br, I;
III) a benzoheterocycle such as benzo[b]furan, indole, N-alkylindole, indazole, benzo[b]thiophene bearing at their 5-position a bis(2-haloethyl)amino) moiety and joined by carboxamide group to a ω-azido polyethylene glycol spacer 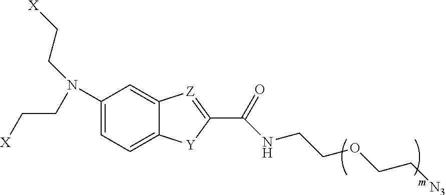
wherein
m is from 1 to 6
X is halogen selected from: Cl, Br, I
Z is —CH— or —N—
Y is —O—, —S—, —NH or N—C₁₋₆alkyl alkyl chain.

8. Ligands of the extra-cellular chromatin for medical use according to the previous claim 3, wherein said DNA alkylating agents are selected in the group comprising: all bis-chloroethylamine or bis-bromoethylamine derivatives such as chlorambucil, norchlorambucil, bendamustin, bromo benzoic mustard or melphalan, Busulfan, N-(2-(2-(2-azidoethoxy)ethoxy)ethyl)-5-(bis(2-chloroethyl)amino)benzofuran-2-carboxamide (MBF) and 4-[Bis-(2-bromoethyl)amino]benzoic acid.

9. Ligands of the extra-cellular chromatin for medical use according to any one of the previous claims, wherein said ligand is selected in the group comprising: Digoxigenin, Distamycin, Ciprofloxacin, Thioridazine, Netropsin, Trabectedin, Sulindac, Piperaquine, Atabrine (Mepacrine), Mitonafide, Chloroquine, Amsacrine, Indomethacin, Methylene blue, berberine, proflavin, daunomycin, doxorubicin, thalidomide, tallimustin, hairpin polyamides, bis(benzimidazole), aureolic acids and bisquaternary ammonium heterocycles) and derivatives of any one of the above.

10. Ligands of the neutrophil extra-cellular traps (NETs) for medical use according to any one of the preceding claims, wherein said treatment comprises the treatment of a condition selected in the group comprising: occlusive vascular conditions.

11. Ligands of the neutrophil extra-cellular traps (NETs) for medical use according to the preceding claim, wherein said occlusive vascular condition leads to acute organ ischemia.

12. Ligands of the neutrophil extra-cellular traps (NETs) for medical use according to any one of the preceding claims 1 to 9, wherein said prevention comprises the prevention of a condition selected in the group comprising: distal embolization and pulmonary embolism.

13. Ligands of the neutrophil extra-cellular traps (NETs), which are used for the adhesion to or the removal of a thrombus.

14. Ligands of the neutrophil extra-cellular traps (NETs) for medical use according to any one of the preceding claims, wherein said ligands are within a substrate.

15. A substrate comprising one or more of the ligands according to any one of claims 1 to 14.

16. A substrate comprising one or more of the ligands according to any one of claims 1 to 8, which is in the form of a coating.

17. The substrate according to the preceding claim 16, wherein said coating comprises one or more layer or a polymer or of a co-polymer.

18. The substrate according to the preceding claim 17, wherein said polymer is selected from the group comprising: polydopamine, PEG-bis-amine and copolymer polydopamine and PEG-bis-amine.

19. The substrate according to any one of the preceding claims 16 to 18, which comprises a secondary coating.

20. The substrate according to the preceding claim, wherein said secondary coating is a saccharidic or a polysaccharidic substrate or is a polyglycolic acid (PGA), polylactic acid (PLA) or polyvinyl alcohol (PVA) substrate.

21. The substrate according to the preceding claim, wherein said secondary coating comprises mannitol.

22. The ligand according to any one of the preceding claims 1 to 14, which is derivatized with a suitable group.

23. The ligand according to any one of the preceding claims 1 to 14, which is derivatized with a suitable derivatization group via a suitable linker.

24. The ligand according to the preceding claim, wherein said linker is selected from the group of the linkers having any one of the following structures:
—(O—CH₂—CH₂)ₙ—
wherein n is from 3 to 5,
or
—(CH₂)ₘ—O—(CH₂)ₙ—
wherein m and n are independently 3 to 7,
or
-[(—CH₂)ₙ—O-]_{z}-(CH₂)ₘ—N₃,
wherein
n is from 1 to 10, preferably from 2 to 6 and more preferably from 2 to 4,
z is from 1 to 6, preferably from 1 to 4 and more preferably from 1 to 3,
m is from 1 to 10 and preferably from 2 to 5.

25. The ligand according to any one of the preceding claims 22 to 24, wherein said suitable derivatization group is represented by azide (—N₃).

26. The ligand according to the preceding claim, wherein the linker has any one of the following structures:

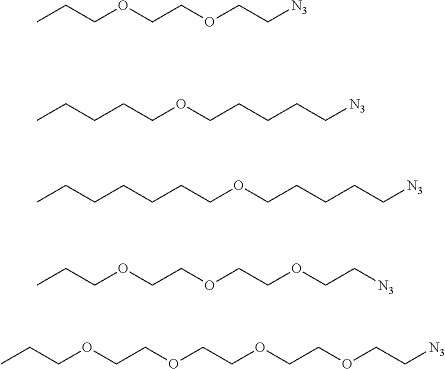 [SMILES: CCCOCCOCCN=[N+]=[N-] ; CCCCCOCCCCCN=[N+]=[N-] ; CCCCCCCOCCCCCN=[N+]=[N-] ; CCCOCCOCCOCCN=[N+]=[N-] ; CCCOCCOCCOCCOCCN=[N+]=[N-]]

27. The ligand according to any one of the preceding claims 22 to 25, which is selected from the group comprising: N-(2-(2-(2-azidoethoxy)ethoxy)ethyl)-5-(bis(2-chloroethyl)amino)benzofuran-2-carboxamide (benzofuran azide mustard, MBF), 4-[Bis-(2-bromoethyl)amino]benzoic acid (BBM), N-(2-(2-(2-azidoethoxy)ethoxy)ethyl)-3-(4-(bis(2-chloroethyl)amino)phenyl)propanamide (PPM, norchlorambucil-azide), N-(2-(2-(2-azidoethoxy)ethoxy)ethyl)-4-(4-(bis(2-chloroethyl)amino)phenyl)butanamide (chlorambucil-azide), 4-(4-(4-((5-azidopentyl)oxy)butyl)piperazin-1-yl)-7-chloroquinone (Piperaquine-azide), 4-[Bis-(2-bromoethyl)amino]benzoic acid (azobenzene-azide, BBM), busulfan-azide (MsA).

28. A process for the preparation of a coating for a device comprising the one or more of the ligands according to any one of claims 15 to 21 comprising a first step for the preparation of a solution or of a suspension of the substrate.

29. A device comprising a portion coated with one or more of the ligands according to any one of claims 1 to 14 of or the substrate of any one of claims 15 to 21.

30. The device comprising a portion coated with the ligand according to any one of claims 1 to 14 or the substrate according to any one of claims 15 to 21, which is selected in the group comprising thrombectomy device, flowretriever, filters, embolic protection devices, distal protection devices, including balloon angioplasty, inferior vena cava filters.

31. The device according to claim 29 or 30 for use in the field of interventional neuroradiology.

32. The device according to any one of claims 29 to 31 for use in a field, which is other than the interventional neuroradiology.

33. A method for the treatment of venous or arterial thromboembolism in a subject comprising the step of contacting a thrombus comprising neutrophil extra-cellular traps (NETs) with a ligand according to any one of claims 1 to 14 or with a substrate according to claim any one of claims 15 to 21.

34. A method for the diagnosis venous or arterial thromboembolism in a subject comprising the step of contacting a thrombus comprising neutrophil extra-cellular traps (NETs) with a ligand according to any one of claims 1 to 14 or with a substrate according to any one of claims 15 to 21.

35. Use of a ligand according to any one of claims 1 to 14 or of a substrate according to any one of claims 15 to 21 for the adhesion to a thrombus comprising neutrophil extra-cellular traps (NETs).

36. Use of a ligand according to any one of claims 1 to 14 or of a substrate according to any one of claims 15 to 21 for the adhesion and the removal of thrombus comprising neutrophil extra-cellular traps (NETs).

37. Use of a ligand according to any one of claims 1 to 14 or of a substrate according to any one of claims 15 to 21 in the field of interventional neuroradiology comprising neutrophil extra-cellular traps (NETs).

38. Use of a ligand according to any one of claims 1 to 14 or of a substrate according to any one of claims 15 to 21 in the field other than interventional neuroradiology.
